(19) United States
(12) Patent Application Publication
(10) Pub. No.: US 2025/0120732 A1
(43) Pub. Date: Apr. 17, 2025

(54) SURGICAL TOOL WITH RELAXED FLEX CIRCUIT ARTICULATION

(71) Applicant: Cilag GmbH International, Zug (CH)

(72) Inventors: Shane R. Adams, Lebanon, OH (US); Matthew D. Cowperthwait, Cincinnati, OH (US); Adam D. Hensel, Gahanna, OH (US); Frederick E. Shelton, IV, Hillsboro, OH (US); Sarah A. Worthington, Cincinnati, OH (US)

(21) Appl. No.: 18/379,776

(22) Filed: Oct. 13, 2023

(51) Int. Cl. A61B 17/28; A61B 17/00; A61B 17/29

(52) U.S. Cl. CPC A61B 17/282; A61B 17/29; A61B 2017/00017; A61B 2017/00398; A61B 2017/2908

(57) ABSTRACT

A surgical instrument includes an end effector comprising first and second jaws, and a longitudinal channel; an articulation joint assembly. The end effector extends along a central longitudinal axis in an unarticulated position, and wherein the end effector is articulatable about an articulation joint toward a fully articulated position on a first side of the unarticulated position. A flex circuit is to transmit at least one of data or power through the articulation joint. The flex circuit is offset from the central longitudinal axis, and comprises a proximal flex-circuit portion positioned in the shaft, a distal flex-circuit portion positioned in the end effector, and an intermediate flex-circuit portion positioned in a cavity defined by the articulation joint assembly. The intermediate flex-circuit portion maintains a relaxed curved state with an unchanged effective length as the end effector is articulated toward the fully articulated position.

### SUMMARY

A surgical instrument comprises a shaft that defines a central longitudinal axis; an end effector comprising a first jaw, a second jaw, and a longitudinal channel; an articulation joint assembly extending between the shaft and the end effector, wherein the end effector extends along the central longitudinal axis in an unarticulated position, and wherein the end effector is articulatable about an articulation joint toward a fully articulated position on a first side of the unarticulated position; and a flex circuit configured to transmit at least one of data or power through the articulation joint, wherein the flex circuit is offset from the central longitudinal axis, and wherein the flex circuit comprises a proximal flex-circuit portion positioned in the shaft, a distal flex-circuit portion positioned in the end effector, and an intermediate flex-circuit portion positioned in a cavity defined by the articulation joint assembly, wherein the intermediate flex-circuit portion maintains a relaxed curved state with an unchanged effective length as the end effector is articulated toward the fully articulated position.

A surgical instrument comprises a shaft that defines a central longitudinal axis; an end effector comprising a first jaw, a second jaw, and a longitudinal channel; an articulation joint assembly extending between the shaft and the end effector, wherein the end effector extends along the central longitudinal axis in an unarticulated position, wherein the end effector is articulatable about an articulation joint toward a fully articulated position on a first side of the unarticulated position; and a flex circuit configured to transmit at least one of data or power through the articulation joint, wherein the flex circuit is offset from the central longitudinal axis, and wherein the flex circuit comprises a proximal flex-circuit portion positioned in the shaft, a distal flex-circuit portion positioned in the end effector, and an intermediate flex-circuit portion extending in a cavity defined by the articulation joint assembly, wherein the intermediate flex-circuit portion remains folded and unstretched as the end effector is articulated toward the fully articulated position, without pulling on the proximal flex-circuit portion or the distal flex-circuit portion.

A surgical instrument comprises a shaft that defines a central longitudinal axis; an end effector comprising a first jaw, a second jaw, and a longitudinal channel configured to accommodate a staple cartridge; an articulation joint assembly extending between the shaft and the end effector, wherein the end effector extends along the central longitudinal axis in an unarticulated position, wherein the end effector is articulatable about an articulation joint toward a fully articulated position on a first side of the unarticulated position; and a flex circuit configured to transmit at least one of data or power through the articulation joint, wherein the flex circuit is offset from the central longitudinal axis, and wherein the flex circuit comprises a proximal flex-circuit portion positioned in the shaft, a distal flex-circuit portion positioned in the end effector, and an intermediate flex-circuit portion positioned in a cavity defined by the articulation joint assembly, wherein the intermediate flex-circuit portion remains folded and relaxed as the end effector is articulated toward the fully articulated position, without pulling on the proximal flex-circuit portion or the distal flex-circuit portion.

### DRAWINGS

Various features of the aspects described herein are set forth with particularity in the appended claims. The various aspects, however, both as to organization, and methods of operation, together with advantages thereof, may be understood in accordance with the following description taken in conjunction with the accompanying drawings as follows:

FIG. 1 is a distal perspective view of a surgical instrument, in accordance with the present disclosure.

FIG. 2 is a partial perspective view of the surgical instrument of FIG. 1 showing a flex circuit, in phantom, extending through an articulation joint assembly, in accordance with the present disclosure.

FIG. 3 is a partial cross section view of the surgical instrument of FIG. 1 showing the flex circuit of FIG. 2.

FIG. 4 illustrates the surgical instrument of FIG. 1 in a first fully articulated position.

FIG. 5 illustrates the surgical instrument of FIG. 1 in an unarticulated position.

FIG. 6 illustrates the surgical instrument of FIG. 1 in a second fully articulated position.

Corresponding reference characters indicate corresponding items throughout the several views.

### DESCRIPTION

Applicant of the present application owns the following U.S. Patent Applications that were filed on even date herewith and which are each herein incorporated by reference in their respective entireties:
U.S. Patent Application, titled METHOD OF OPERATING A SURGICAL STAPLING INSTRUMENT; Attorney Docket No. END9484USNP2/220491-2M;
U.S. Patent Application, titled SURGICAL STAPLING SYSTEMS WITH ADAPTIVE STAPLE FIRING ALGORITHMS; Attorney Docket No. END9484USNP3/220491-3;
U.S. Patent Application, titled LEARNED TRIGGERS FOR ADAPTIVE CONTROL OF SURGICAL STAPLING SYSTEMS; Attorney Docket No. END9484USNP4/220491-4;
U.S. Patent Application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. Patent Application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. Patent Application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. Patent Application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. Patent Application, titled WIRING HARNESS FOR SMART STAPLER WITH MULTI AXIS ARTICULATION; Attorney Docket No. END9484USNP10/220491-10;
U.S. Patent Application, titled SURGICAL SYSTEM WITH WIRELESS ARRAY FOR POWER AND DATA TRANSFER; Attorney Docket No. END9484USNP11/220491-11; and
U.S. Patent Application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. Patent Applications that were filed on even date herewith and which are each herein incorporated by reference in their respective entireties:
U.S. Patent Application, titled METHOD OF ASSEMBLING A STAPLE CARTRIDGE; Attorney Docket No. END9484USNP13/220491-13M;
U.S. Patent Application, titled CONTROL SURFACES ON A STAPLE DRIVER OF A SURGICAL STAPLE CARTRIDGE; Attorney Docket No. END9484USNP14/220491-14;
U.S. Patent Application, titled INTEGRAL CARTRIDGE STIFFENING FEATURES TO REDUCE CARTRIDGE DEFLECTION; Attorney Docket No. END9484USNP15/220491-15;
U.S. Patent Application, titled STAPLE CARTRIDGE COMPRISING WALL STRUCTURES TO REDUCE CARTRIDGE DEFLECTION; Attorney Docket No. END9484USNP16/220491-16;
U.S. Patent Application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. Patent Application, titled STAPLE CARTRIDGE COMPRISING A SLED HAVING A DRIVER LIFT CAM; Attorney Docket No. END9484USNP18/220491-18;
U.S. Patent Application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. Patent Application, titled STAPLE CARTRIDGE COMPRISING A COMPOSITE SLED; Attorney Docket No. END9484USNP20/220491-20;
U.S. Patent Application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. Patent Application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. Patent Application, titled DUAL INDEPENDENT KEYED LOCKING MEMBERS ACTING ON THE SAME DRIVE MEMBER; Attorney Docket No. END9484USNP23/220491-23;
U.S. Patent Application, titled ADJUNCTS FOR USE WITH SURGICAL STAPLING INSTRUMENTS; Attorney Docket No. END9484USNP24/220491-24;
U.S. Patent Application, titled ADJUNCTS FOR USE WITH SURGICAL STAPLING INSTRUMENTS; Attorney Docket No. END9484USNP25/220491-25;
U.S. Patent Application, titled JAW CONTROL SURFACES ON A SURGICAL INSTRUMENT JAW; Attorney Docket No. END9484USNP26/220491-26;
U.S. Patent Application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. Patent Application, titled STAPLE CARTRIDGES COMPRISING TRACE RETENTION FEATURES; Attorney Docket No. END9484USNP29/220491-29; and
U.S. Patent Application, titled STAPLE CARTRIDGES COMPRISING STAPLE RETENTION FEATURES; Attorney Docket No. END9484USNP30/220491-30.

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment”, or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation. Such modifications and variations are intended to be included within the scope of the present invention.

The terms “proximal” and “distal” are used herein with reference to a clinician manipulating the handle portion of the surgical instrument. The term “proximal” referring to the portion closest to the clinician and the term “distal” referring to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various methods, instruments, and systems are provided for performing surgical procedures. Various surgical systems disclosed herein include working portions that can be inserted into a body in any way, such as through a natural orifice, through an incision or puncture hole formed in tissue, etc. The working portions or end effector portions can be inserted directly into a patient's body or can be inserted through an access device that has a working channel. As the present Detailed Description proceeds, it will be understood that the various unique and novel arrangements of the various forms of surgical systems disclosed herein may be effectively employed in connection with robotically-controlled surgical systems and/or hand-held surgical systems. Various robotic systems, instruments, components and methods are disclosed in U.S. patent application Ser. No. 13/118,241, entitled SURGICAL STAPLING INSTRUMENTS WITH ROTATABLE STAPLE DEPLOYMENT ARRANGEMENTS, which is incorporated by reference herein in its entirety.

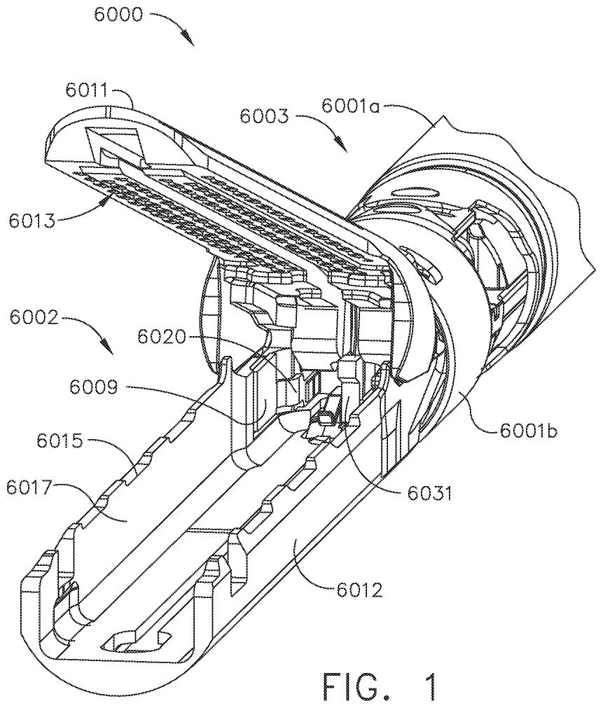

FIG. 1 is a distal perspective view of a surgical instrument 6000, in accordance with the present disclosure. The surgical instrument 6000 includes a shaft 6001 that defines a central longitudinal axis 6005 extending therethrough. The shaft 6001 includes a proximal shaft portion 6001a that can be coupled to a handle in instances where the surgical instrument is handheld. In other instances, the proximal shaft portion 6001a is coupled to a robotic arm for use with a surgical robot, for example.

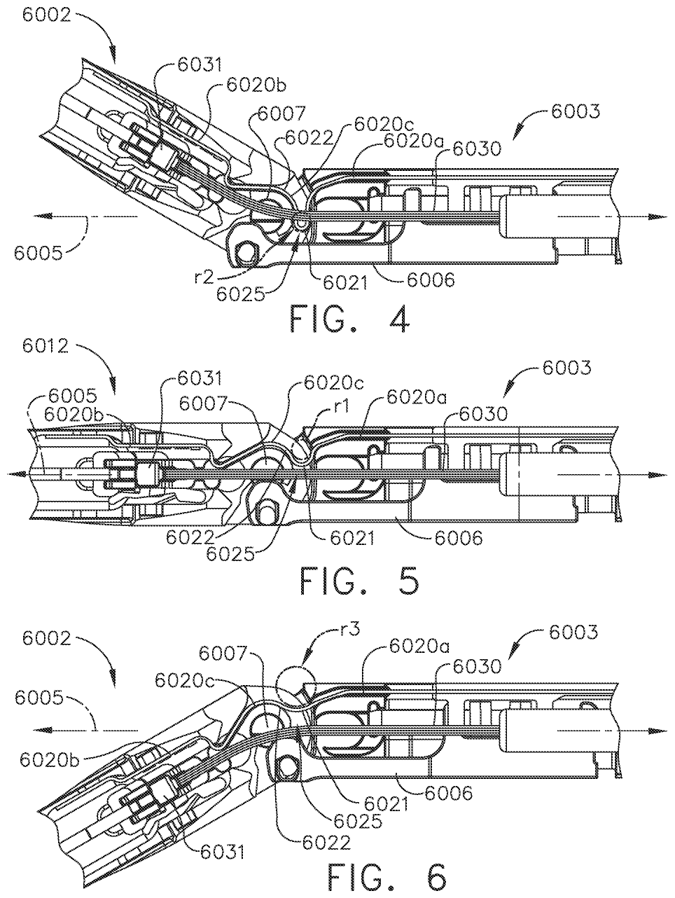

The shaft 6001 further includes a distal shaft portion 6001b coupled to an end effector 6002. An articulation joint assembly 6003 extends between the proximal shaft portion 6001a and the distal shaft portion 6001b. The articulation joint assembly 6003 includes an articulation driver 6006 movable distally to rotate the end effector 6002 from an unarticulated position, as illustrated in FIG. 5, toward a first fully articulated position, as illustrated in FIG. 4, about an articulation joint 6007. Furthermore, the articulation driver 6006 is movable proximally to rotate the end effector 6002 from the unarticulated position, as illustrated in FIG. 5, toward a second fully articulated position, as illustrated in FIG. 6, about the articulation joint 6007. In the illustrated example, the first fully articulated position is on a first side of the unarticulated position, and the second fully articulated position is on a second side of the unarticulated position, opposite the first side, for example.

Further to the above, the end effector 6002 includes a first jaw 6011 and a second jaw 6012 movable relative to the first jaw 6011 to transition the end effector 6002 from an open configuration, as illustrated in FIG. 1, to a closed configuration to grasp tissue between the first jaw 6011 and the second jaw 6012. In the illustrated example, the first jaw 6011 comprises an anvil including staple forming pockets 6013 and the second jaw 6012 includes a longitudinal channel 6015 configured to receive a staple cartridge 6014 including staples deformable against the staple forming pockets 6013 of the anvil. In accordance with the present disclosure, the first jaw 6011 and the second jaw 6012 may be equipped with electrodes configured to deliver therapeutic and/or non-therapeutic energy to the grasped tissue.

In the illustrated example, a drive shaft 6030 extends distally along the longitudinal central axis 6005 in the unarticulated position, and is coupled to a firing beam 6031 movable by the drive shaft 6030 to drive a sequential deployment of staples from the staple cartridge 6014 and to cut the stapled tissue. Additional details are described in U.S. Patent Application Ser. No. 14/200,111, entitled CONTROL SYSTEMS FOR SURGICAL INSTRUMENTS, filed Mar. 7, 2014, which issued on Apr. 25, 2017 as U.S. Pat. No. 9,629,629, which is hereby incorporated by reference herein in its entirety.

The surgical instrument 6000 further includes a flex circuit 6020 that transmits at least one of data or power through the articulation joint assembly 6003 to the end effector 6002 from a power source or a data source proximal to the articulation joint assembly 6003. In accordance with the present disclosure, the flex circuit 6020 may transmit power to the end effector 6002 for powering an electronics package 6009 that communicates with a chip in the staple cartridge 6014, for example. Additionally, or alternatively, the flex circuit 6020 may define a communication pathway between the chip and a processor of the surgical instrument 6000 positioned proximal to the articulation joint assembly 6003. In accordance with the present disclosure, as illustrated in FIG. 1, the electronics package 6009 can be fixed to a side wall 6017 of the longitudinal channel 6015.

In accordance with the present disclosure, the flex circuit 6020 may comprise floating ends. Additionally, in accordance with the present disclosure, the flex circuit 6020 may comprise fixed ends. Alternatively, in accordance with the present disclosure, the flex circuit 6020 may comprise one floating end and one fixed end. Further, in accordance with the present disclosure, the flex circuit 6020 may include a flexible substrate, and a conductive layer disposed on the flexible substrate.

Translating a flex circuit around an articulation joint requires some manner of accommodation for the different distances spanned by the flex circuit in the unarticulated position and the articulated position. One approach is to provide some manner of strain-relief or allow the flex circuit to change its effective length. A key challenge with strain relief is the number of times the strain relief can be activated and the number of times it can fully recover either due to loss of elasticity, interference due to debris, or fatigue of the copper wiring within the flex circuit. Also, changing the effective length of the flex circuit has its own challenges. The unaccommodated additional length, in the unarticulated position, may cause portions of the flex circuit to extend outside the surgical instrument, or may wrap around, or interfere, with other components.

In accordance with the present disclosure, the present disclosure may present solutions that avoid the forgoing challenges. In accordance with the present disclosure, as illustrated in FIGS. 4-6, the flex circuit 6020 can remain folded in the unarticulated position (FIG. 5), the first articulated position (FIG. 4), and the second articulated position (FIG. 6). The planned folding of the flex circuit maintains the flex circuit 6020 in a relaxed curved state during a full range of articulation of the end effector 6002, while transitioning between different degrees of curvature without fully straightening or needing strain relief. As discussed in greater detail below, the flex circuit 6020 never reaches a fully unfolded configuration throughout the full range of articulation of the end effector 6002, thereby nullifying any need for a refold biasing force.

In the illustrated example, the flex circuit 6020 is offset from the central longitudinal axis 6005, and includes a proximal flex-circuit portion 6020a positioned in the proximal shaft portion 6001a, a distal flex-circuit portion 6020b positioned in the distal shaft portion 6001b, and an intermediate flex-circuit portion 6020c extending between the proximal and distal flex-circuit portions 6020a, 6020b through the articulation joint assembly 6003 in a predefined passageway, as illustrated in FIGS. 4-6. The intermediate flex-circuit portion 6020c includes a proximal segment 6021 folded into a cavity 6025 defined in the articulation joint assembly 6003. In the illustrated example, the cavity 6025 is situated proximal to the articulation joint 6007. The intermediate flex-circuit portion 6020c further includes a distal segment 6022 extending distally from the folded proximal segment 6021. The distal segment 6022 partially wraps around the articulation joint 6007 and, then, extends distally toward the distal flex-circuit portion 6020b.

The folded proximal segment 6021 is tucked in the cavity 6025, and transitions between a first folded configuration, as illustrated in FIG. 5, and a second folded configuration, as illustrated in FIG. 4. In the second folded configuration, the proximal segment 6021 extends across the drive shaft 6030 and across the central longitudinal axis 6005. When the end effector 6002 is returned to the unarticulated position (FIG. 5) from the first articulated position (FIG. 4), the proximal segment 6021 retreats to a less curved, or less folded, configuration but remains in a folded/curved state without straightening out completely, thus, ensuring that the intermediate flex-circuit portion 6020c remains in a relaxed state as the end effector 6002 repeatedly transitions between the unarticulated position (FIG. 5) and the first articulated position (FIG. 4).

Further to the above, the proximal segment 6021 transitions between the second folded configuration, as illustrated in FIG. 5, and a third folded configuration, as illustrated in FIG. 6. I and transitions between a first folded configuration, as illustrated in FIG. 5, and a second folded configuration, as illustrated in FIG. 4. In the second folded configuration, the proximal segment 6021 extends across the drive shaft 6030 and across the central longitudinal axis 6005. When the end effector 6002 is returned to the unarticulated position (FIG. 5) from the first articulated position (FIG. 4), the proximal segment 6021 retreats to a less curved, or less folded, configuration but remains in a folded/curved state without straightening out completely, thus, ensuring that the intermediate flex-circuit portion 6020c remains in a relaxed state as the end effector 6002 repeatedly transitions between the unarticulated position (FIG. 5) and the first articulated position (FIG. 4). When the end effector 6002 is transitioned from the unarticulated position (FIG. 5) to the second articulated position (FIG. 6), the proximal segment 6021 retreats to a less curved, or less folded, configuration but remains in a folded/curved state without straightening out completely, thus, ensuring that the intermediate flex-circuit portion 6020c remains in a relaxed state as the end effector 6002 repeatedly transitions between the unarticulated position (FIG. 5) and the second articulated position (FIG. 6)

Accordingly, the proximal segment 6021 maintains a curved, or folded, state throughout a full range of articulation of the end effector 6002, while experiencing a reduction in curvature as the end effector 6002 moves from the first articulated position (FIG. 4) to the second articulated position (FIG. 6). Said another way, the proximal segment 6021 comprises a first curvature in the first articulated position (FIG. 4) greater than a second curvature of the proximal segment 6021 in the unarticulated position (FIG. 5), and a third curvature in the second articulated position (FIG. 6) less than the second curvature and the first curvature.

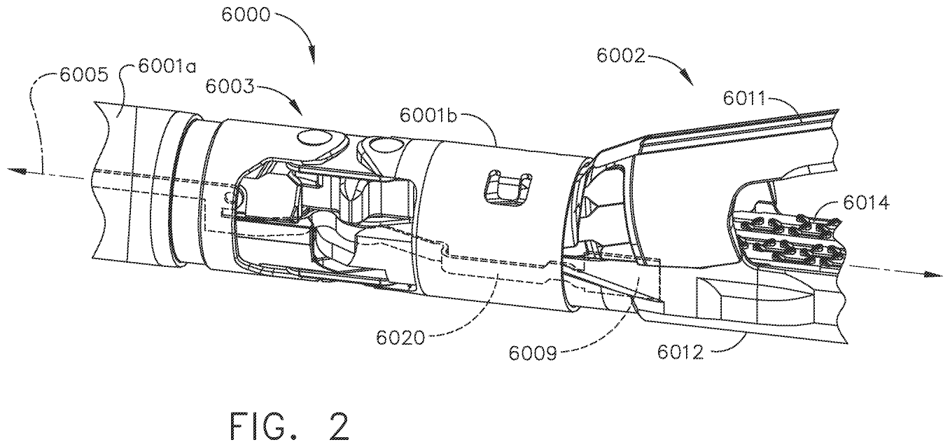

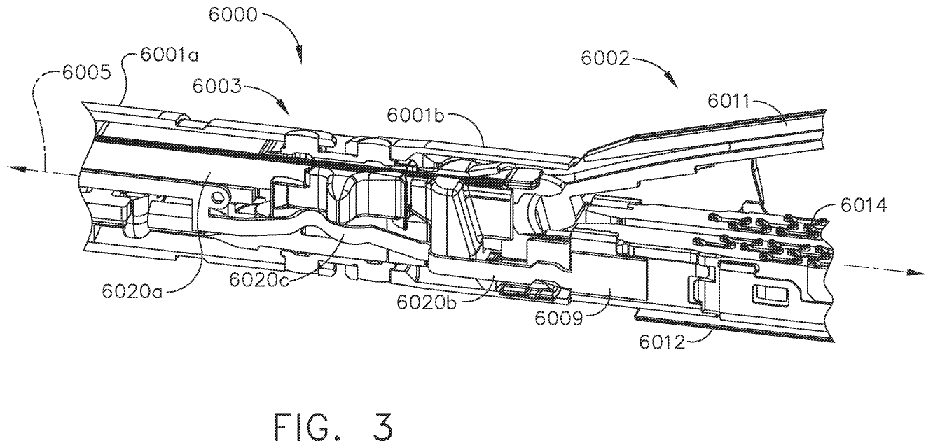

As best illustrated in FIGS. 2 and 3, the flex circuit 6020 comprises a flat ribbon shape generally extending longitudinally alongside the drive shaft 6030, and including a thickness, and a width greater than the thickness. The proximal segment 6022 of the intermediate flex-circuit portion 6020c is formed by folding the flex circuit 6020 about an axis extending along the width, which permits the proximal segment 6021 to be tucked into the cavity 6025. The different curvatures of the proximal segment 6021 comprise different radii of curvatures that extend along the thickness of the proximal segment 6021.

In accordance with the present disclosure, proximal segment 6021 may have a flex radius greater than, or equal to, about 2× the thickness of the flex circuit 6020 throughout a full range of articulation of the end effector 6002. Further, in accordance with the present disclosure, the proximal segment 6021 may have a flex radius selected from a range of about 2× to about 4× the thickness of the flex circuit 6020, for example, throughout a full range of articulation of the end effector 6002. This arrangement ensures an articulation of the end effector 6002 that only causes a change in the tortuous path of the flex circuit 6020 without strain relief or a change in the effective length of the intermediate flex-circuit portion 6002c.

In the illustrated example, the proximal segment 6021 defines a first radius of curvature (r1) in the unarticulated position (FIG. 5), a second radius of curvature (r2) in the first articulated position (FIG. 4), and a third radius of curvature (r3) in the second articulated position (FIG. 6). The radii of curvature (r1, r2, r3) are different from one another, but all permit the proximal segment 6021 to remain within the cavity 6025 as the proximal segment transitions therebetween.

Further to the above, referring primarily to FIGS. 4-6, the length of the proximal segment 6021 changes as the end effector 6002 transitions between the first articulation position (FIG. 4), the unarticulated position (FIG. 5), and the second articulated position (FIG. 6), without fully straitening, or while maintaining a curves/folded state throughout its full range of articulation. The excess length of the proximal segment 6021, which is tucked in the cavity 6025, permits a relaxed transition between the different positions of the end effector without requiring strain relief, and without pulling on the proximal and/or distal flex circuit portions 6002a, 6002b.

In the illustrated example, the proximal segment 6021 enters the cavity 6025 by extending toward the drive shaft 6030, then curves away from the drive shaft 6030. The distal segment 6022, which extends distally from the proximal segment 6021, and partially wraps around the articulation joint 6077, then extends distally toward the end effector 6002. Other pathways and curvatures of the intermediate flex-circuit portion 6002c are contemplated by the present disclosure.

Examples of the apparatus and method according to various aspects of the present disclosure are provided below in the following numbered clauses. An aspect of the apparatus and method may include any one or more than one, and any combination of, the numbered clauses described below.

Clause 1—A surgical instrument (6000) comprises a shaft (6001) that defines a central longitudinal axis (6005); an end effector (6002) comprising a first jaw (6011), a second jaw (6012), and a longitudinal channel (6015); an articulation joint assembly (6003) extending between the shaft and the end effector, wherein the end effector extends along the central longitudinal axis in an unarticulated position, and wherein the end effector is articulatable about an articulation joint (6007) toward a fully articulated position on a first side of the unarticulated position; and a flex circuit (6020) configured to transmit at least one of data or power through the articulation joint, wherein the flex circuit is offset from the central longitudinal axis, and wherein the flex circuit comprises a proximal flex-circuit portion (6020a) positioned in the shaft, a distal flex-circuit portion (6020b) positioned in the end effector, and an intermediate flex-circuit portion (6020c) positioned in a cavity (6025) defined by the articulation joint assembly, wherein the intermediate flex-circuit portion maintains a relaxed curved state with an unchanged effective length as the end effector is articulated toward the fully articulated position.

Clause 2—The surgical instrument of Clause 1, wherein the intermediate flex-circuit portion comprises a first curvature in the unarticulated position, and a second curvature in the fully articulated position, wherein the intermediate flex-circuit portion is to transition from the first curvature toward the second curvature as the end effector is articulated toward the fully articulated position.

Clause 3—The surgical instrument of Clause 2, wherein the intermediate flex-circuit portion is to transition toward the first curvature as the end effector is articulated toward the unarticulated position.

Clause 4—The surgical instrument of any of Clauses 1-3, wherein the longitudinal channel comprises a side wall (6017), and wherein the distal flex-circuit portion is fixed to the side wall.

Clause 5—The surgical instrument of any of Clauses 1-4, wherein the flex circuit defines a thickness, and a width greater than the thickness, wherein the intermediate flex-circuit portion is formed by folding the flex circuit about an axis extending along the width.

Clause 6—The surgical instrument of any of Clauses 1-5, wherein the flex circuit defines a thickness, and a width greater than the thickness, wherein the intermediate flex-circuit portion defines a first radius of curvature (r1) in the unarticulated position, and wherein the intermediate flex-circuit portion defines a second radius of curvature (r2), different than the first radius of curvature, in the fully articulated position.

Clause 7—The surgical instrument of Clause 6, wherein the first radius of curvature and the second radius of curvature extend along the thickness.

Clause 8—The surgical instrument of any of Clauses 6-7, wherein the first radius of curvature is less than the second radius of curvature, and wherein the second radius of curvature is greater than or equal to double the thickness.

Clause 9—The surgical instrument of any of Clauses 1-8, wherein the intermediate flex-circuit portion moves deeper into the cavity as the end effector is articulated from the unarticulated position toward the fully articulated position.

Clause 10—The surgical instrument of any of Clauses 1-9, wherein the articulation joint assembly comprises the articulation joint, and wherein the cavity is proximal to the articulation joint.

Clause 11—The surgical instrument of any of Clauses 1-10, further comprising a drive shaft (6030), wherein the cavity extends laterally across the drive shaft.

Clause 12—The surgical instrument of any of Clauses 1-11, wherein the curved portion moves toward the drive shaft as the end effector is articulated from the unarticulated position toward the fully articulated position.

Clause 13—The surgical instrument of any of Clauses 1-12, wherein the fully articulated position is a first fully articulated position, and wherein the end effector is articulatable about the articulation joint toward a second fully articulated position on a second side of the unarticulated position opposite the first side.

Clause 14—The surgical instrument of Clause 13, wherein the intermediate flex- circuit portion extends through a predefined passageway defined in the articulation joint assembly, and wherein the intermediate flex-circuit portion is longer than the predefined passageway.

Clause 15—The surgical instrument of any of Clauses 13-14, wherein the intermediate flex-circuit portion defines a first curvature in the unarticulated position, a second curvature in the first fully articulated position, wherein the intermediate flex-circuit portion is to transition from the first curvature toward the second curvature as the end effector is articulated toward the first fully articulated position, and a third curvature in the second fully articulated position, wherein the first curvature and the second curvature are different than the third curvature.

Clause 16—A surgical instrument (6000) comprises a shaft (6001) that defines a central longitudinal axis (6005); an end effector (6002) comprising a first jaw (6011), a second jaw (6012), and a longitudinal channel (6015); an articulation joint assembly (6003) extending between the shaft and the end effector, wherein the end effector extends along the central longitudinal axis in an unarticulated position, wherein the end effector is articulatable about an articulation joint (6007) toward a fully articulated position on a first side of the unarticulated position; and a flex circuit (6020) configured to transmit at least one of data or power through the articulation joint, wherein the flex circuit is offset from the central longitudinal axis, and wherein the flex circuit comprises a proximal flex-circuit portion (6020a) positioned in the shaft, a distal flex-circuit portion (6020b) positioned in the end effector, and an intermediate flex-circuit portion (6020c) positioned in a cavity (6025) defined by the articulation joint assembly, wherein the intermediate flex-circuit portion remains folded and unstretched as the end effector is articulated toward the fully articulated position, without pulling on the proximal flex-circuit portion or the distal flex-circuit portion.

Clause 17—A surgical instrument (6000) comprises a shaft (6001) that defines a central longitudinal axis (6005); an end effector (6002) comprising a first jaw (6011), a second jaw (6012), and a longitudinal channel (6015) configured to accommodate a staple cartridge (6014); an articulation joint assembly (6003) extending between the shaft and the end effector, wherein the end effector extends along the central longitudinal axis in an unarticulated position, wherein the end effector is articulatable about an articulation joint (6007) toward a fully articulated position on a first side of the unarticulated position; and a flex circuit (6020) configured to transmit at least one of data or power through the articulation joint, wherein the flex circuit is offset from the central longitudinal axis, and wherein the flex circuit comprises a proximal flex-circuit portion (6020a) positioned in the shaft, a distal flex-circuit portion (6020b) positioned in the end effector, and an intermediate flex-circuit portion (6020c) positioned in a cavity (6025) defined by the articulation joint assembly, wherein the intermediate flex-circuit portion remains folded and relaxed as the end effector is articulated toward the fully articulated position, without pulling on the proximal flex-circuit portion or the distal flex-circuit portion.

Clause 18—A surgical instrument comprises a shaft that defines a central longitudinal axis; an end effector comprising a first jaw, a second jaw, and a longitudinal channel; an articulation joint assembly extending between the shaft and the end effector, wherein the end effector extends along the central longitudinal axis in an unarticulated position, and wherein the end effector is articulatable about an articulation joint toward a fully articulated position on a first side of the unarticulated position; and a flex circuit configured to transmit at least one of data or power through the articulation joint, wherein the flex circuit is offset from the central longitudinal axis, and wherein the flex circuit comprises a proximal flex-circuit portion positioned in the shaft, a distal flex-circuit portion positioned in the end effector, and an intermediate flex-circuit portion positioned in a cavity defined by the articulation joint assembly, wherein the intermediate flex-circuit portion maintains a relaxed curved state with an unchanged effective length as the end effector is articulated toward the fully articulated position.

Clause 19—The surgical instrument of Clause 18, wherein the intermediate flex-circuit portion comprises a first curvature in the unarticulated position, and a second curvature in the fully articulated position, wherein the intermediate flex-circuit portion is to transition from the first curvature toward the second curvature as the end effector is articulated toward the fully articulated position.

Clause 20—The surgical instrument of Clause 19, wherein the intermediate flex-circuit portion is to transition toward the first curvature as the end effector is articulated toward the unarticulated position.

Clause 21—The surgical instrument of Clause 18, wherein the longitudinal channel comprises a side wall, and wherein the distal flex-circuit portion is fixed to the side wall.

Clause 22—The surgical instrument of Clause 18, wherein the flex circuit defines a thickness, and a width greater than the thickness, wherein the intermediate flex-circuit portion is formed by folding the flex circuit about an axis extending along the width.

Clause 23—The surgical instrument of Clause 18, wherein the flex circuit defines a thickness, and a width greater than the thickness, wherein the intermediate flex-circuit portion defines a first radius of curvature in the unarticulated position, and wherein the intermediate flex-circuit portion defines a second radius of curvature, different than the first radius of curvature, in the fully articulated position.

Clause 24—The surgical instrument of Clause 23, wherein the first radius of curvature and the second radius of curvature extend along the thickness.

Clause 25—The surgical instrument of Clause 24, wherein the first radius of curvature is less than the second radius of curvature, and wherein the second radius of curvature is greater than or equal to double the thickness.

Clause 26—The surgical instrument of Clause 18, wherein the intermediate flex-circuit portion moves deeper into the cavity as the end effector is articulated from the unarticulated position toward the fully articulated position.

Clause 27—The surgical instrument of Clause 18, wherein the articulation joint assembly comprises the articulation joint, and wherein the cavity is proximal to the articulation joint.

Clause 28—The surgical instrument of Clause 18, further comprising a drive shaft, wherein the cavity extends laterally across the drive shaft.

Clause 29—The surgical instrument of Clause 18, wherein the intermediate flex- circuit portion moves toward a drive shaft as the end effector is articulated from the unarticulated position toward the fully articulated position.

Clause 30—The surgical instrument of Clause 18, wherein the fully articulated position is a first fully articulated position, and wherein the end effector is articulatable about the articulation joint toward a second fully articulated position on a second side of the unarticulated position opposite the first side.

Clause 31—The surgical instrument of Clause 30, wherein the intermediate flex-circuit portion extends through a predefined passageway defined in the articulation joint assembly, and wherein the intermediate flex-circuit portion is longer than the predefined passageway.

Clause 32—The surgical instrument of Clause 18, wherein the fully articulated position is a first fully articulated position, and wherein the intermediate flex-circuit portion defines a first curvature in the unarticulated position, a second curvature in the first fully articulated position, wherein the intermediate flex-circuit portion is to transition from the first curvature toward the second curvature as the end effector is articulated toward the first fully articulated position, and a third curvature in the second fully articulated position, wherein the first curvature and the second curvature are different than the third curvature.

Clause 33—A surgical instrument comprises a shaft that defines a central longitudinal axis; an end effector comprising a first jaw, a second jaw, and a longitudinal channel; an articulation joint assembly extending between the shaft and the end effector, wherein the end effector extends along the central longitudinal axis in an unarticulated position, wherein the end effector is articulatable about an articulation joint toward a fully articulated position on a first side of the unarticulated position; and a flex circuit configured to transmit at least one of data or power through the articulation joint, wherein the flex circuit is offset from the central longitudinal axis, and wherein the flex circuit comprises a proximal flex-circuit portion positioned in the shaft, a distal flex-circuit portion positioned in the end effector, and an intermediate flex-circuit portion extending in a cavity defined by the articulation joint assembly, wherein the intermediate flex-circuit portion remains folded and unstretched as the end effector is articulated toward the fully articulated position, without pulling on the proximal flex-circuit portion or the distal flex-circuit portion.

Clause 34—The surgical instrument of Clause 33, wherein the intermediate flex-circuit portion comprises a first curvature in the unarticulated position, and a second curvature in the fully articulated position, wherein the intermediate flex-circuit portion is to transition from the first curvature toward the second curvature as the end effector is articulated toward the fully articulated position.

Clause 35—The surgical instrument of Clause 33, wherein the flex circuit defines a thickness, and a width greater than the thickness, wherein the intermediate flex-circuit portion is formed by folding the flex circuit about an axis extending along the width

Clause 36—A surgical instrument comprises a shaft that defines a central longitudinal axis; an end effector comprising a first jaw, a second jaw, and a longitudinal channel configured to accommodate a staple cartridge; an articulation joint assembly extending between the shaft and the end effector, wherein the end effector extends along the central longitudinal axis in an unarticulated position, wherein the end effector is articulatable about an articulation joint toward a fully articulated position on a first side of the unarticulated position; and a flex circuit configured to transmit at least one of data or power through the articulation joint, wherein the flex circuit is offset from the central longitudinal axis, and wherein the flex circuit comprises a proximal flex-circuit portion positioned in the shaft, a distal flex-circuit portion positioned in the end effector, and an intermediate flex-circuit portion positioned in a cavity defined by the articulation joint assembly, wherein the intermediate flex-circuit portion remains folded and relaxed as the end effector is articulated toward the fully articulated position, without pulling on the proximal flex-circuit portion or the distal flex-circuit portion.

Clause 37—The surgical instrument of Clause 36, wherein the intermediate flex-circuit portion comprises a first curvature in the unarticulated position, and a second curvature in the fully articulated position, wherein the intermediate flex-circuit portion is to transition from the first curvature toward the second curvature as the end effector is articulated toward the fully articulated position.

Various embodiments described herein are described in the context of staples removably stored within staple cartridges for use with surgical stapling instruments. In some circumstances, staples can include wires which are deformed when they contact an anvil of the surgical stapler. Such wires can be comprised of metal, such as stainless steel, for example, and/or any other suitable material. Such embodiments, and the teachings thereof, can be applied to embodiments which include fasteners removably stored with fastener cartridges for use with any suitable fastening instrument.

Various embodiments described herein are described in the context of linear end effectors and/or linear fastener cartridges. Such embodiments, and the teachings thereof, can be applied to non-linear end effectors and/or non-linear fastener cartridges, such as, for example, circular and/or contoured end effectors. For example, various end effectors, including non-linear end effectors, are disclosed in U.S. patent application Ser. No. 13/036,647, filed Feb. 28, 2011, entitled SURGICAL STAPLING INSTRUMENT, now U.S. Pat. No. 8,561,870, which is hereby incorporated by reference in its entirety. Additionally, U.S. patent application Ser. No. 12/893,461, filed Sep. 29, 2012, entitled STAPLE CARTRIDGE, now U.S. Pat. No. 8,733,613, is hereby incorporated by reference in its entirety. U.S. patent application Ser. No. 12/031,873, filed Feb. 15, 2008, entitled END EFFECTORS FOR A SURGICAL CUTTING AND STAPLING INSTRUMENT, now U.S. Pat. No. 7,980,443, is also hereby incorporated by reference in its entirety. U.S. Pat. No. 8,393,514, entitled SELECTIVELY ORIENTABLE IMPLANTABLE FASTENER CARTRIDGE, which issued on Mar. 12, 2013, is also hereby incorporated by reference in its entirety. One skilled in the art will recognize that the herein described components (e.g., operations), devices, objects, and the discussion accompanying them are used as examples for the sake of conceptual clarity and that various configuration modifications are contemplated. Consequently, as used herein, the specific examples set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Although various embodiments have been described herein, many modifications, variations, substitutions, changes, and equivalents to those embodiments may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed embodiments. The following claims are intended to cover all such modification and variations.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical application to thereby enable one of ordinary skill in the art to utilize the various embodiments and with various modifications as are suited to the particular use contemplated. It is intended that the claims submitted herewith define the overall scope.

It is worthy to note that any reference numbers included in the appended claims are used to reference exemplary embodiments/elements described in the present disclosure. Accordingly, any such reference numbers are not meant to limit the scope of the subject matter recited in the appended claims.

1-17. (canceled)

18. A surgical instrument, comprising:
a shaft that defines a central longitudinal axis;
an end effector, comprising:
a first jaw;
a second jaw; and
a longitudinal channel;
an articulation joint assembly extending between the shaft and the end effector, wherein the end effector extends along the central longitudinal axis in an unarticulated position, and wherein the end effector is articulatable about an articulation joint toward a fully articulated position on a first side of the unarticulated position; and
a flex circuit to transmit at least one of data or power through the articulation joint, wherein the flex circuit is offset from the central longitudinal axis, and wherein the flex circuit comprises:
a proximal flex-circuit portion positioned in the shaft;
a distal flex-circuit portion positioned in the end effector; and
an intermediate flex-circuit portion positioned in a cavity defined by the articulation joint assembly, wherein the intermediate flex-circuit portion maintains a relaxed curved state with an unchanged effective length as the end effector is articulated toward the fully articulated position.

19. The surgical instrument of claim 18, wherein the intermediate flex-circuit portion comprises:
a first curvature in the unarticulated position; and
a second curvature in the fully articulated position, wherein the intermediate flex-circuit portion is to transition from the first curvature toward the second curvature as the end effector is articulated toward the fully articulated position.

20. The surgical instrument of claim 19, wherein the intermediate flex-circuit portion is to transition toward the first curvature as the end effector is articulated toward the unarticulated position.

21. The surgical instrument of claim 18, wherein the longitudinal channel comprises a side wall, and wherein the distal flex-circuit portion is fixed to the side wall.

22. The surgical instrument of claim 18, wherein the flex circuit defines
a thickness; and
a width greater than the thickness, wherein the intermediate flex-circuit portion is formed by folding the flex circuit about an axis extending along the width.

23. The surgical instrument of claim 18, wherein the flex circuit defines:
a thickness; and
a width greater than the thickness, wherein the intermediate flex-circuit portion defines a first radius of curvature in the unarticulated position, and wherein the intermediate flex-circuit portion defines a second radius of curvature, different than the first radius of curvature, in the fully articulated position.

24. The surgical instrument of claim 23, wherein the first radius of curvature and the second radius of curvature extend along the thickness.

25. The surgical instrument of claim 24, wherein the first radius of curvature is less than the second radius of curvature, and wherein the second radius of curvature is greater than or equal to double the thickness.

26. The surgical instrument of claim 18, wherein the intermediate flex-circuit portion moves deeper into the cavity as the end effector is articulated from the unarticulated position toward the fully articulated position.

27. The surgical instrument of claim 18, wherein the articulation joint assembly comprises the articulation joint, and wherein the cavity is proximal to the articulation joint.

28. The surgical instrument of claim 18, further comprising a drive shaft, wherein the cavity extends laterally across the drive shaft.

29. The surgical instrument of claim 18, wherein the intermediate flex-circuit portion moves toward a drive shaft as the end effector is articulated from the unarticulated position toward the fully articulated position.

30. The surgical instrument of claim 18, wherein the fully articulated position is a first fully articulated position, and wherein the end effector is articulatable about the articulation joint toward a second fully articulated position on a second side of the unarticulated position opposite the first side.

31. The surgical instrument of claim 30, wherein the intermediate flex-circuit portion extends through a predefined passageway defined in the articulation joint assembly, and wherein the intermediate flex-circuit portion is longer than the predefined passageway.

32. The surgical instrument of claim 18, wherein the intermediate flex-circuit portion defines:
a first curvature in the unarticulated position;
a second curvature in the first fully articulated position, wherein the intermediate flex-circuit portion is to transition from the first curvature toward the second curvature as the end effector is articulated toward the first fully articulated position; and
a third curvature in the second fully articulated position, wherein the first curvature and the second curvature are different than the third curvature.

33. A surgical instrument, comprising:
a shaft that defines a central longitudinal axis;
an end effector, comprising:
a first jaw;
a second jaw; and
a longitudinal channel;
an articulation joint assembly extending between the shaft and the end effector, wherein the end effector extends along the central longitudinal axis in an unarticulated position, wherein the end effector is articulatable about an articulation joint toward a fully articulated position on a first side of the unarticulated position; and
a flex circuit configured to transmit at least one of data or power through the articulation joint, wherein the flex circuit is offset from the central longitudinal axis, and wherein the flex circuit comprises:
a proximal flex-circuit portion positioned in the shaft;
a distal flex-circuit portion positioned in the end effector; and
an intermediate flex-circuit portion extending in a cavity defined by the articulation joint assembly, wherein the intermediate flex-circuit portion remains folded and unstretched as the end effector is articulated toward the fully articulated position, without pulling on the proximal flex-circuit portion or the distal flex-circuit portion.

34. The surgical instrument of claim 33, wherein the intermediate flex-circuit portion comprises a first curvature in the unarticulated position, and a second curvature in the fully articulated position, wherein the intermediate flex-circuit portion is to transition from the first curvature toward the second curvature as the end effector is articulated toward the fully articulated position.

35. The surgical instrument of claim 33, wherein the flex circuit defines a thickness, and a width greater than the thickness, wherein the intermediate flex-circuit portion is formed by folding the flex circuit about an axis extending along the width.

36. A surgical instrument, comprising:
a shaft that defines a central longitudinal axis;
an end effector, comprising:
a first jaw;
a second jaw; and
a longitudinal channel configured to accommodate a staple cartridge;
an articulation joint assembly extending between the shaft and the end effector, wherein the end effector extends along the central longitudinal axis in an unarticulated position, wherein the end effector is articulatable about an articulation joint toward a fully articulated position on a first side of the unarticulated position; and
a flex circuit configured to transmit at least one of data or power through the articulation joint, wherein the flex circuit is offset from the central longitudinal axis, and wherein the flex circuit comprises:
a proximal flex-circuit portion positioned in the shaft;
a distal flex-circuit portion positioned in the end effector; and
an intermediate flex-circuit portion positioned in a cavity defined by the articulation joint assembly, wherein the intermediate flex-circuit portion remains folded and relaxed as the end effector is articulated toward the fully articulated position, without pulling on the proximal flex-circuit portion or the distal flex-circuit portion.

37. The surgical instrument of claim 36, wherein the intermediate flex-circuit portion comprises a first curvature in the unarticulated position, and a second curvature in the fully articulated position, wherein the intermediate flex-circuit portion is to transition from the first curvature toward the second curvature as the end effector is articulated toward the fully articulated position.
